(19) United States
(12) Patent Application Publication
(10) Pub. No.: US 2004/0152085 A1
(43) Pub. Date: Aug. 5, 2004

(54) Surface for collection and/or purification of nucleic acids

(75) Inventors: Kathy Terlesky, Charlottesville, VA (US); Chuck Daitch, Ann Arbor, MI (US); Chris Earle, Ruckersville, VA (US); Eric Van Gieson, Charlottesville, VA (US)

Correspondence Address: MILLEN, WHITE, ZELANO & BRANIGAN, P.C., 2200 CLARENDON BLVD., SUITE 1400, ARLINGTON, VA, 22201, US

(73) Assignee: Veridian Systems Division, Arlington, VA

(21) Appl. No.: 10/357,388

(22) Filed: Feb. 4, 2003

(51) Int. Cl. C12Q 1/68; C12M 1/34

(52) U.S. Cl. 435/6; 435/287.2

(57) ABSTRACT

A substrate for collecting nucleic acids, for example, DNA, (and processes of making and using the same), comprising a surface; an aerogel coated on said surface; an active silane attached so said aerogel; and a nucleic acid binding agent attached to said silane.

The invention was made with government support under contract # TSWG 157113-0041-0001, Sweepstakes 00-VIS-3726-00100 and Sweepstakes 00-VIS-3726-00800. The government has certain rights in this invention.

The prior art uses several different techniques to collect nucleic acids, for example, DNA, one of which is the use of methidium-spermine-sepharose beads. Methidium-spermine intercalates double stranded nucleic acids and is removed from the mixture by removing the sepharose bead supports. Other known nucleic acid collection techniques involve binding nucleic acid electrostatically to a charged surface. The impurities attached to such a surface during such collection are generally washed away. Commercially available kits are in the market and include “Qiagen” and “Wizard” and other anion exchange based matrices. However, these and other prior art methods are in need of improvement.

Desirable for instance, are substrates having the mechanical properties necessary to accommodate high-throughput collection processes.

In one aspect, the invention is directed to a method and apparatus for the sampling, collection and/or purification of nucleic acids, such as DNA and/or RNA, preferably double stranded DNA, from aqueous samples. Nucleic acids in solution bind to surfaces according to the invention under a variety of environmental conditions, for example, under a variety of salinity, temperature and/or pH conditions, and are removable from said surfaces by selective chemical treatment or heat.

In another aspect, the invention is directed to a method and apparatus for the removal and decontamination of nucleic acids from biopharmaceutical purification systems. For instance, the removal of all nucleic acids following fermentation or cell culture is critical prior to releasing waste that may contain genetically-modified nucleic acids in the waste stream.

An advantage of the invention is that nucleic acids remain bound to the surface under conditions of high salt concentrations. Another advantage is the invention works well on samples collected from the environment. The invention also does not collect proteins and other biological molecules which have an overall negative charge.

The present invention utilizes a surface, preferably a smooth solid surface, such as that of a glass bead, a microscope slide, any other glass surface, or that of a plastic, which is coated with an aerogel to increase the surface area of the surface. The aerogel coating is then silanated with an activated silane, for example, with a silane that contains one or more groups, such as, amino, thiol, isocyanato, carboxyl, and/or alcohol groups, preferably amino groups, which allow for the attachment of nucleic acid binders, e.g., DNA binding molecules, for example, intercalating agents and/or minor-groove binding molecules, which include, for example, SYBR and psoralen. The active groups, for example, the amino, thiol, etc., groups listed above, can be attached to an alkyl group, such as propyl, which is attached to a silicon atom.

The surfaces prepared according to the invention bind a significant amount of nucleic acids from solution under a variety of salinity conditions, including conditions of high salt concentration, for example, 1 molar NaCl, and higher. The nucleic acids can be released from these surfaces by any means that disrupts the process of nucleic acid binding, such as intercalation, typically by means of heat (above the Tm of the nucleic acids, which is specific to a nucleic acid's strand length and sequence) or chemical denaturation, for example, by a detergent or alcohol, or by raising the pH, which will cause the double strands to separate, i.e., denature, and thus disrupting the intercalation. Another method of releasing the nucleic acids from the substrate is by the use of electrophoretic methods. Electrophoretic methods apply an eletrophoretic current, and may be used in combination with mild temperature and salt concentration conditions and facilitate use in chromatographic methods. In the case of electrophoretic methods, it is preferable that the solid surface be made of glass, which is amenable to electrophoresis.

Optionally, if it is desired to increase the distance from the surface of the aerogel to the intercalator, linker groups, and preferably linker groups that are useful in affinity chromatography, which are generally known in the art, can be inserted between the silane and the intercalator. In such a case, the linker can bind to the active group of the silane and can also bind to the intercalator, i.e., the linker is bifunctional. The linker's functional groups can be any group that is capable of leading to a bond between the noted compounds; however, they are typically groups selected from those discussed above with respect to the active groups in the silane. Any known linker group or groups can be used in the invention. Exemplary non-limiting linkers are: N-Boc-1,3-diaminopropane, N-Boc-1,4-diaminobutane, N-Boc-1,5-diaminopentane, N-Boc-1,6-diaminohexane, 3-(Boc-amino)-1-propanol, 4-(Boc-amino)-1-butanol, 5-(Boc-amino)-1-pentanol, 6-(Boc-amino)-1-hexanol, Na-Boc-L-lysine, NE-Boc-L-lysine, and Na-Boc-L-serine methyl ester and many more.

An advantage of the invention is that nucleic acids, for example, DNA, can be selectively removed from other biological molecules, under a variety of conditions in solution, and at a variety of concentrations, and then discarded on a collection matrix or selectively released for further analysis, purification, or amplification. In addition, by using solid substrates such as glass for the aerogel/silane/intercalator or other binder formulation, the construct can withstand large shear forces and hydrostatic pressures. This feature is important for high throughput nucleic acid collection devices and in chromatographic methods where high flow-rates and pressure drops may be present.

The nucleic acids can also be amplified directly on the substrates of the invention by, for example, polymerase chain reaction. Polymerase chain reaction, PCR, is a well known biochemical technique that amplifies or makes multiple copies of a single nucleic acid, for example, a DNA molecule. To amplify, for example, a double stranded DNA (dsDNA), the dsDNA is denatured by raising the temperature above the Tm, i.e., the temperature at which the double strands separate to give two single stranded DNA (ssDNA) compliments. The DNA is then cooled slightly and the PCR primers (short strands of complimentary DNA) anneal to the ssDNA. With these primers in place, DNA polymerase can begin to copy the strands by adding the complimentary bases to the ssDNA to form a dsDNA copy. The process is then repeated multiple times to obtain multiple copies from the original dsDNA on the substrates of the invention. Preferably, the substrates of the invention after nucleic acids have attached thereto, are added to a PCR reaction mixture to amplify any DNA that the substrates have bound. Nucleic acids, for example, double stranded DNA, bound to the substrates of the invention can be added directly to a PCR mixture, amplified, then separated and detected by gel electrophoresis.

The substrates to be modified for use in the methods and products of the present invention include materials that have, or can be modified to have, thereon an aerogel coating surface. Suitable substrates are preferably inorganic materials, including but not limited to silicon, glass, silica, diamond, quartz, alumina, silicon nitride, platinum, gold, aluminum, tungsten, titanium, various other metals and various other ceramics. Alternatively, polymeric materials such as polyesters, polyamides, polyimides, acrylics, polyethers, polysulfones, fluoropolymers, etc. may be used as suitable organic substrates. The substrate used may be provided in any suitable form or size, such as slides, wafers, fibers, beads, particles, strands, precipitates, gels, sheets, tubing, spheres, containers, capillaries, pads, slices, films, plates, slides, etc. The substrate may have any convenient shape, such as that of a disc, square, sphere, circle, etc. The support can further be fashioned as a bead, dipstick, test tube, pin, membrane, channel, capillary tube, column, or as an array of pins or glass fibers. Glass is the preferred solid substrate, preferably in the form of beads.

Aerogels are known in the art, and any of them without limitations can be used in the presently claimed invention. Aerogels can be applied to the surface of the substrates by a variety of means, which are not limited, for example, by known dipping and coating methods. Aerogels are a type of sol-gel. Preferred are silicon-based aerogels which are preferably not doped.

Unger, et al., in U.S. Pat. No. 6,444,660, teach that the term “aerogel” refers to generally spherical or spheroidal entities which are characterized by a plurality of small internal voids. The aerogels may be formulated from synthetic materials (for example, a foam prepared from baking resorcinol and formaldehyde), as well as natural materials, such as carbohydrates (polysaccharides) or proteins. See also Abbott, et al., in U.S. Pat. No. 6,277,489, teaching that aerogels are characterized by accessible, cylindical, branched mesopores having high porosity and low density. Aerogels are typically formed by the controlled condensation of small (polymeric or colloidal) particles. Agglomeration of the particles is controlled by chemical processes, usually the sol-gel process. The use of this process to form aerogels is well-known in the art. See, for example, Husing, et al, Angewandte Chemie (International Edition in English), 37: 23-45 (1998), and U.S. Pat. No. 6,447,991, which are entirely incorporated herein by reference.

'991 teaches a sol-gel process where a solution of silicate monomer (sol) undergoes polymerization to a gel. Specifically, an ethanol solution of tetraethoxysilane Si(OCH₂CH₃)₄ in the presence of water, ethanol, and catalyst, undergoes partial hydrolysis and a condensation reaction to form a sol (a colloidal dispersion of particles in liquid). As the process of polymerization continues, a solid silicate network separates out of the solution (gel point). The solid is still “soaking” in the ethanol solution; this biphasic system is usually referred to as the alcogel. Subsequent removal of the liquid phase from the alcogel by supercritical drying, results in a low density, highly porous silica aerogel. Various regimes of pore size evolve during polymerization, e.g., 2-100 nm, but smaller and larger values are also applicable. Statistical control over the evolution and distribution of pore size can be accomplished by varying reaction conditions, such as pH, solvent, temperature, hydrolysis ratio, and monomer concentration.

Other methods for producing aerogels include, supercritical drying of liquid from a wet gel comprising particulate material. A solvent containing the particulate material is put into its supercritical state. Typically, the wet gel is placed in an autoclave and covered with additional solvent. After the autoclave is closed, the temperature is slowly raised resulting in an increase in pressure. Both the temperature and the pressure are adjusted to values above the supercritical point of the solvent and kept there for a period of time. Once the autoclave is completely filled with the solvent, the solvent is then slowly vented at constant temperature, resulting in a pressure drop. When ambient pressure is reached, the autoclave is cooled to room temperature and opened. Preferred solvents include, alcohols, acetone, 2-propanol, carbon dioxide and water.

An aerogel layer can generally range in thickness from a monomolecular thickness to about several hundred microns, however, a thickness of about 1 micrometer ±0.2 micrometers is preferred.

The term “silane” or “silicone” is understood in its conventional meaning and has one or more active groups that are available to attach to an intercalator, etc., and/or a linker if one is present. Preferably, the silane contains one silicon atom; however, polymeric silane groups, i.e., silane groups that have more than one silicon atom, are within the scope of the invention. The silane preferably contains one or two active groups when it has one silicon atom, however, more than one or two are within the scope of the invention, especially when the silane contains more than one silicon atom.

The silanes attach to the aerogel coating in the same manner as they would attach to glass in standard well-known silane chemistry. The silanes useful for the invention can bind to the aerogel's hydroxyl groups and include a wide variety of silanes, preferably amino silanes, such as amino alkyl silanes, or amino alkoxy silanes, including silanes having more than one amino group. U.S. Pat. No. 6,441,159 teaches typical silane chemistry for the attachment of silane to a surface. The silanol (Si—OH) groups in the aerogel backbone undergo a condensation reaction with the hydrolyzed silane, for example, alkoxysilane leading to a covalent bond between the silane and the aerogel surface. A group, for example, propylamine, is then available for further reaction with the linker or directly with the intercalator or other binders.

Intercalators are also known in the art, and any of them without limitation can be used in the presently claimed invention. The intercalator, however, has to have a suitable binding group to accomplish the attachment to a group of the silane attached to the aerogel surface or the linker, such as, to an amine group, via amide bonding, for example. Intercalators lacking such groups can be activated by known chemical techniques. Typical intercalating agents are: ethidium, ethidium bromide, methidium, acridine, aminoacridine, acridine orange and derivatives therof, psoralen, proflavin, ellipticine, actinomycin D, daunomycin, malachite green, phenyl neutral red, mitomycin C, HOECHST 33342, HOECHST 33258, aclarubicin, DAPI, SYBR, Adriamycin, pirarubicin, actinomycin, tris(phenanthroline) zinc salt, tris(phenanthroline) ruthenium salt, tris(phenantroline) cobalt salt, di(phenanthroline) zinc salt, di(phenanthroline) ruthenium salt, di(phenanthroline) cobalt salt, bipyridine platinum salt, terpyridine platinum salt, phenanthroline platinum salt tris(bipyridyl) zinc salt, tris(bipyridyl) ruthenium salt, tris(bipyridyl) cobalt salt di(bipyridyl) zinc salt, di(bipyridyl) ruthenium salt, di(bipyridyl) cobalt salt, etc.

The term “intercalator” describes the insertion of planar aromatic or heteroaromatic compounds between adjacent base pairs of double stranded nucleic acids, e.g., DNA (dsDNA). The intercalating agents are characterized by their tendency to intercalate specifically to double stranded nucleic acid such as double stranded DNA. Some intercalating agents have in their molecules a flat intercalating group such as a phenyl group, which intercalates between the base pairs of the double stranded nucleic acid, whereby binding to the double stranded nucleic acid. Most of the intercalating agents are optically active and some of them are used in qualification of nucleic acids. Certain intercalating agents exhibit electrode response. Therefore, determination of physical change, especially optical or electrochemical change, may serve to detect the intercalating agents bound to a double stranded nucleic acid.

Electrochemically or optically active intercalating agents are, but are not limited to, ethidium, ethidium bromide, acridine, aminoacridine, acridine orange, proflavin, ellipticine, actinomycin D, daunomycin, mitomycin C, HOECHST 33342, HOECHST 33258, aclarubicin, DAPI, Adriamycin, pirarubicin, actinomycin, tris(phenanthroline) zinc salt, tris(phenanthroline) ruthenium salt, tris(phenantroline) cobalt salt, di(phenanthroline) zinc salt, di(phenanthroline) ruthenium salt, di(phenanthroline) cobalt salt, bipyridine platinum salt, terpyridine platinum salt, phenanthroline platinum salt, tris(bipyridyl) zinc salt, tris(bipyridyl) ruthenium salt, tris(bipyridyl) cobalt salt, di(bipyridyl) zinc salt, di(bipyridyl) ruthenium salt, di(bipyridyl) cobalt salt, and the like. Other intercalating agents are those listed in Published Japanese Patent Application No. 62-282599.

In addition to the intercalating agents which are reversibly reacted themselves during oxidation-reduction reaction as listed above, the determination of electrochemical change using an electrode may employ a metal complex containing as a center metal a substance capable of undergoing electrically reversible oxidation-reduction reaction, namely, a metallo intercalator. Such metallo intercalators include for example tris(phenanthroline) zinc salt, tris(phenanthroline) ruthenium salt, tris(phenanthroline) cobalt salt, di(phenthroline) zinc salt, di(phenanthroline) ruthenium salt, di(phenanthroline) cobalt salt, bipyridine cobalt salt, terpyridine platinum salt, phenanthroline platinum salt, tris(bipyridyl) zinc salt, tris(bipyridyl) ruthenium salt, tris(bipyridyl) cobalt salt, di(bipyridyl) zinc salt, di(bipyridyl) ruthenium salt, di(bipyridyl) cobalt salt and the like.

When conducting the detection of a nucleic acid, e.g., a gene using an electrode, an intercalating agent exhibiting electrochemiluminescence may also be employed. Such intercalating agents are, but are not limited to, for example, luminol, lucigenin, pyrene, diphenylanthracene rubrene and acridinium derivaties. The electrochemiluminescene of the intercalating agents listed above may be enhanced by the enhancers such as luciferin derivatives such as firefly luciferin and dihydroluciferin, phenols such as phenyl phenol and chlorophenol as well as naphthols.

Optical signals generated by the electrochemiluminescence may directly be detected from the solution using, for example, a photocounter. Alternatively, an optical fiber electrode produced by forming a transparent electrode at the tip of an optical fiber may also be used to detect the signal indirectly.

Fluorescent dyes are also suitable for detecting nucleic acids. For example, ethidium bromide is an intercalating agent that displays increased fluorescence when bound to double stranded DNA rather than when in free solution. Ethidium bromide can be used to detect both single and double stranded nucleic acids, although the affinity of ethidium bromide for single stranded nucleic acid is relatively low.

Preferred intercalator agents are psoralen, SYBR, ethidium, ethidium bromide, methidium, actinomycin, malachite green, phenyl neutral red, derivatives of acridine, more preferred among these are psoralen and SYBR. The nature of SYBR's interaction with DNA is not exactly known. Some in the art believe it is a minor groove binder. However, knowing its exact mode of interaction with the DNA is not relevant to the practice of the invention.

Modified intercalators are commercially available, such is psoralen and SYBR, and/or can be prepared by well known methods in the art. The modification should be such that a group on the intercalator should be available to lead to a bond between the modified group in the silane or to the modified group in the linker.

Other nucleic acid binders can also be used in the invention instead of the intercalators, such as groove binder moieties, for example, minor groove binder moieties. The minor groove binder moiety according to U.S. Pat. No. 5,801,155 is a radical of a molecule having a molecular weight of approximately 150 to approximately 2000 Daltons which molecule binds in a non-intercalating manner into the minor groove of double stranded DNA, RNA or hybrids thereof with an association constant greater than approximately 10³M⁻¹. However, some minor groove binders bind to the high affinity sites of double stranded DNA with an association constant of the magnitude of 10⁷ to 10⁹ M⁻¹.

Gjerde, et al., in U.S. Pat. No. 6,210,885 describes reversible DNA-binding dyes, such as chromophore molecules which reversibly bind by direct interaction with the edges of base pairs in either of the grooves (major or minor) of nucleic acids. These dyes are non-intercalative DNA binding agents. Non-limiting examples of DNA groove binding dyes include Netropsin (N′-(2-amidinoethyl)-4-(2-guanidinoacetamido)-1,1′-dimethyl-N,4′-bi[pyrrol e-2-carboxamide]) (Sigma), Hoechst dye no. 33258 (Bisbenzimide, B-2261, Sigma), Hoechst dye no. 33342, (Bisbenzimide, B2261, Sigma), and Hoechst dye no. 2495 (Benzoxanthene yellow, B-9761, Sigma). Preferred reversible DNA-binding dyes in the present invention include fluorescent dyes. Non-limiting examples of reversible DNA-binding dyes include PICO GREEN (P-7581, Molecular Probes), ethidium bromide (E-8751, Sigma), propidium iodide (P-4170, Sigma), Acridine orange (A-6014, Sigma), 7-aminoactinomycin D (A-1310, Molecular Probes), cyanine dyes (e.g., TOTO, YOYO, BOBO, and POPO), SYTO, SYBR Green I, SYBR Green II, SYBR DX, OliGreen, CyQuant GR, SYTOX Green, SYTO9, SYTO10, SYTO17, SYBR14, FUN-1, DEAD Red, Hexidium Iodide, Dihydroethidium, Ethidium Homodimer, 9-Amino-6-Chloro-2-Methoxyacridine, DAPI, DIPI, Indole dye, Imidazole dye, Actinomycin D, Hydroxystilbamidine, and LDS 751. Numerous reversible DNA-binding dyes are described in Handbook of Fluorescent Probes and Research Chemicals, Ch. 8.1 (1997) (Molecular Probes, Inc.); European Patent Application No. EP 0 634 640 A1; Canadian Patent No. CA 2,119,126; and in the following U.S. Pat. Nos. 5,410,030; 5,321,130; 5,432,134; 5,445,946; 4,716,905.

Some minor groove binding molecules can be covalently bound to an oligoneucleotide. A minor groove binder is a molecule that binds within the minor groove of double stranded deoxyribonucleic acid (DNA). Although a general chemical formula for all known minor groove binding compounds cannot be provided because such compounds have widely varying chemical structures, compounds which are capable of binding in the minor groove of DNA, generally speaking, have a crescent shape three dimensional structure. Most minor groove binding compounds of the prior art have a strong preference for A-T (adenine and thymine) rich regions of the B form of double stranded DNA. The minor groove binding compounds, or more accurately stated moieties of the oligonucleotide-minor groove binding conjugates of the present invention, also have the same preference. Nevertheless, minor groove binding compounds which would show preference to C-G (cytosine and guanine) rich regions are also possible.

Examples of known minor groove binding compounds are netropsin, distamycin and lexitropsin, mithramycin, chromomycin A.sub.3, olivomycin, anthramycin, sibiromycin, as well as further related antibiotics and synthetic derivatives. Certain bisquarternary ammonium heterocyclic compounds, diarylamidines such as pentamidine, stilbamidine and berenil, CC-1065 and related pyrroloindole and indole polypeptides, Hoechst 33258, 4′-6-diamidino-2-phenylindole (DAPI) as well as a number of oligopeptides consisting of naturally occurring or synthetic amino acids.

In addition to molecular structures which cause minor groove binding, the minor groove binder moiety may also carry additional functions, as long as those functions do not interfere with minor groove binding ability. For example a reporter group, which makes the minor groove binder readily detectable by color, UV spectrum or other readily discernible physical or chemical characteristic, may be covalently attached to the minor groove binder moiety. An example for such a reporter group is a diazobenzene function which is attached to a carbonyl function of the minor groove binder through a —HN(CH₂)ₘ COO(CH₂)ₘ S(CH₂)ₘ—bridge.

A third category of DNA-binding molecules that can be used in the invention includes molecules that have both groove-binding and intercalating properties. DNA-binding molecules that have both intercalating and minor groove binding properties include actinomycin D, echinomycin, triostin A, and luzopeptin. In general, these molecules have one or two planar polycyclic moieties and one or two cyclic oligopeptides. Luzopeptins, for instance, contain two substituted quinoline chromophores linked by a cyclic decadepsipeptide. They are closely related to the quinoxaline family, which includes echinomycin and triostin A, although they luzopeptins have ten amino acids in the cyclic peptide, while the quinoxaline family members have eight amino acids.

In addition to the major classes of DNA-binding molecules, there are also some small inorganic molecules that can be used in the invention as the nucleic acid binding agent, such as cobalt hexamine, which is known to induce Z-DNA formation in regions that contain repetitive GC sequences (Gessner et al.). Another example is cisplatin, cis-di-amminedichloroplatinum(II), which is a widely used anticancer therapeutic. Cisplatin forms a covalent intrastrand crosslink between the N7 atoms of adjacent guanosines (Rice, et al.). Additionally, U.S. Pat. No. 5,093,963 reports many therapeutic DNA-binding molecules, such as disdamycin, which may be useful in the present invention to replace as intercalating agent.

The invention thus relates to a substrate for collecting and/or purifying nucleic acids, comprising a surface, an aerogel coated onto the surface, an active silane attached to said aerogel, and a nucleic acid binding agent attached to said silane. In another embodiment of the invention, said nucleic acid binding agent is bound to said silicon by a linker group. Preferably, the nucleic acid binding agent binds DNA or RNA, preferably DNA, more preferably double stranded DNA. Preferably, the nucleic acid binding agent is an intercalating agent, or a minor groove binder, more preferably an intercalating agent. Preferably, the active group on the silane is one or more, preferably one or two, amine groups, whereby the intercalating agent attaches to the silane via an amide bond. Preferably, the surface is glass, preferably a glass bead or slide. Preferably, the nucleic acid binding agent is SYBR or psoralen. The invention also relates to a process for preparing the substrates of the invention. The process comprises coating the surface with an aerogel, silanating the aerogel, optionally linking a linker group to the silane, and attaching a nucleic acid binding agent to the silane directly or through an optional linker group.

The invention also relates to a method of collecting or to a method of sampling nucleic acids, preferably, DNA or RNA, comprising bringing into contact a substrate of the invention with a sample from which nucleic acids are to be separated. Preferably, the sample is an aqueous sample. The method can further comprise removing the nucleic acids attached to the substrate by disrupting the bond between the nucleic acid and the nucleic acid binding agent. Preferably, the disruption of the bond is achieved by chemical treatment, heat and/or electrophoretic current.

The invention also relates to a sampling device or a collection device for the collection of nucleic acids comprising a substrate of the invention.

The invention also relates to a chromatography column comprising a substrate of the invention.

The invention additionally relates to a method for removing nucleic acids and/or decontaminating nucleic acids from a biopharmaceutical purification system comprising bringing into contact a substrate of the invention with a solution in a biopharmaceutical purification system that contains nucleic acids. Preferably, the solution is a product of a fermentation process or a cell culture.

The invention also relates to substrates wherein the nucleic acid binding agent has both intercalating and minor groove binding properties, or is a major groove binder, or is an inorganic molecule that binds to nucleic acids or is a therapeutic DNA binding molecule.

Having described the invention, the following example is given to illustrate specific applications of the invention. The specific example is not intended to limit the scope of the invention described in this application. Without further elaboration, it is believed that one skilled in the art can, using the preceding description, utilize the present invention to its fullest extent. The following preferred specific embodiment is, therefore, to be construed as merely illustrative, and not limitative of the remainder of the disclosure in any way whatsoever.

In the foregoing and in the following examples, all temperatures are set forth uncorrected in degrees Celsius; and, unless otherwise indicated, all parts and percentages are by weight.

The entire disclosures of all applications, patents and publications, cited above or below, are hereby incorporated by reference.

### EXAMPLE

Procedure for preparing a substrate according to the invention which has aerogel coated glass beads having an intercalating agent attached to them via 3-aminopropyltrimethoxysilane.

Bead Preparation:

100 μm glass beads are washed first with 50:50 methanol:HCl, rinsed with water, and then washed with 50% aqueous sulfuric acid. The beads are then rinsed with water until the filtrate as a pH of 7 and air dried.

Aerogel Coating of Beads:

An aerogel sol-gel solution is prepared by polymerizing tetraethoxy orthosilicate under basic conditions. The dried beads are then placed in the aerogel sol-gel solution and agitated on a rotary spinner for 12 hours. The beads are removed from the solution by filtration and cured at 100° C. for 60 min.

Silanization of Beads:

The beads are then added to a 3% solution of 3-aminopropyltrimethoxysilane in toluene and agitated on a rotary spinner for 12 hours. The beads are removed from the solution by filtration, rinsed with toluene, and cured at 100° C. for 60 min.

Intercalator Modification:

The beads are then added to a 50 mM solution of succinimidyl-(4-(psoralen-8-yloxy))butyrate in ethanol and agitated on a rotary spinner for 12 hours. The beads are removed from the solution by filtration, rinsed with ethanol, and finally rinsed with PBS buffer.

From the foregoing description, one skilled in the art can easily ascertain the essential characteristics of this invention and, without departing from the spirit and scope thereof, can make various changes and modifications of the invention to adapt it to various usages and conditions.

1. A substrate for collecting nucleic acids comprising a surface; an aerogel on said surface; a silane attached so said aerogel; and a nucleic acid binding agent attached to said silane.

2. A substrate according to claim 1, wherein said nucleic acid binding agent is bound to said silane by a linker group.

3. A substrate according to claim 1, wherein the nucleic acids are DNA or RNA.

4. A substrate according to claim 1, wherein the nucleic acids are double stranded DNA.

5. A substrate according to claim 1, wherein the nucleic acid binding agent is an intercalating agent.

6. A substrate according to claim 1, wherein the nucleic acid binding agent is a minor groove binding agent.

7. A substrate according to claim 1, wherein the silane is an amino silane.

8. A substrate according to claim 5, wherein the intercalating agent is attached to the amino group of the silane via an amide bond.

9. A substrate according to claim 1, wherein the surface is glass or plastic.

10. A substrate according to claim 9, wherein the surface is a glass bead or a microscope slide.

11. A substrate according to claim 10, wherein the surface is a glass bead.

12. A substrate according to claim 1, wherein the nucleic acid binding agent is psoralen or SYBR.

13. A process for preparing a substrate of claim 1, comprising coating a surface with an aerogel, silanating the aerogel, optionally linking a linker group to the silane, and attaching a nucleic acid binding agent to the silane directly or through an optional linker group.

14. A method for collecting nucleic acids comprising bringing into contact a substrate of claim 1, with a sample from which nucleic acids are to be separated.

15. A method according to claim 14, wherein the nucleic acids are DNA or RNA.

16. A method according to claim 14, wherein the sample is an aqueous solution.

17. A method of claim 14, further comprising removing nucleic acids attached to said substrate by disrupting the bond of the nucleic acid binding agent to the nucleic acid.

18. A method according to claim 17, wherein nucleic acids are removed by chemical treatment, heat and/or an electrophoretic current.

19. A sampling device for the collection of nucleic acids comprising a substrate according to claim 1.

20. A chromatography column comprising a substrate according to claim 1.

21. A method of performing chromatography comprising using a substrate according to claim 1 as the chromatography media.

22. A method of sampling nucleic acids comprising collecting nucleic acids by contacting a test sample which may contain said nucleic acids with a substrate according to claim 1.

23. A method for removing nucleic acids and/or decontaminating nucleic acids from a solution obtained from a biopharmaceutical purification system comprising bringing into contact a substrate of claim 1 with said solution.

24. A method according to claim 23, wherein said solution is a product of a fermentation process or a cell culture.

25. A method according to claim 22 further comprising amplifying said nucleic acids.

26. A method according to claim 23 further comprising amplifying said nucleic acids.

27. A method according to claim 25, wherein polymerase chain reaction is used to amplify the nucleic acids.

28. A method according to claim 26, wherein polymerase chain reaction is used to amplify the nucleic acids.
